(19) United States
(12) Patent Application Publication
(10) Pub. No.: US 2021/0045651 A1
(43) Pub. Date: Feb. 18, 2021

(54) APPARATUS AND METHOD FOR DETECTING LIVING BODY AND SYSTEM

(71) Applicant: FUJITSU LIMITED, Kawasaki-shi (JP)

(72) Inventors: Hongchun LI, Beijing (CN); Lili XIE, Beijing (CN); Qian ZHAO, Beijing (CN); Jun TIAN, Beijing (CN)

(73) Assignee: FUJITSU LIMITED, Kawasaki-shi (JP)

(21) Appl. No.: 16/916,610

(22) Filed: Jun. 30, 2020

(30) Foreign Application Priority Data
Aug. 16, 2019 (CN) 201910758078.2

(51) Int. Cl. A61B 5/05; A61B 5/00; A61B 5/11; A61B 5/0205

(52) U.S. Cl. CPC A61B 5/0507; A61B 5/0205; A61B 5/11; A61B 5/7257

(57) ABSTRACT

Embodiments of this disclosure provide an apparatus and method for detecting a living body. The apparatus is to obtain range FFT signals within a first time range according to reflected signals of a microwave radar within the first time range; obtain amplitude distributions and/or phase distributions of distances in the first time range according to the range FFT signals within the first time range, and calculate amplitude fluctuations of the distances within the first time range. A Fourier transform is performed on the amplitude distributions and/or the phase distributions to obtain amplitude spectra and/or phase spectra; and based on magnitudes of the amplitude fluctuations, it may be determined whether there exists a living body corresponding to the distances according to the amplitude spectra and/or the phase spectra, and the amplitude fluctuations, or it may be determined whether there exists a living body corresponding to the distances according to the amplitude spectra and/or the phase spectra.

### CROSS-REFERENCE TO RELATED APPLICATIONS

This application claims priority under 35 USC 119 to Chinese patent application no. 201910758078.2, filed on Aug. 16, 2019, in the China National Intellectual Property Administration, the entire contents of which are incorporated herein by reference.

### TECHNICAL FIELD

This disclosure relates to the field of information technologies.

### BACKGROUND

Monitoring vital signs such as breathing and heartbeat helps to understand the physical health of the human body. In medicine, people obtains breathing and heartbeat information of patients by using professional medical equipment such as electrocardiographs, stethoscopes. With the advancement of technologies, a large number of wearable devices have appeared. People can monitor changes of their physical indicators at all times in daily lives by using smart watches, smart bracelets and other devices. However, the promotion and application of wearable devices are facing problems such as low wearing comfort, and frequent charging.

In recent years, non-contact vital sign detection methods have appeared, such as a microwave radar-based vital sign detection method, which collects microwave signals reflected by a detection object for detection of breathing and heartbeat through the microwave radar. This method has good user experiences and high levels of acceptance, and has broad application prospects.

To apply this method of vital sign detection, a distance between a living body and a microwave radar needs to be determined first.

It should be noted that the above description of the background is merely provided for clear and complete explanation of this disclosure and for easy understanding by those skilled in the art. And it should not be understood that the above technical solution is known to those skilled in the art as it is described in the background of this disclosure.

### SUMMARY

It was found by the inventors that based on the existing methods for detecting a living body, a stationary living body is unable to be differentiated from other stationary objects, and interference caused by noises is unable to be eliminated, resulting in poor accuracy and reliability of detection results.

Embodiments of this disclosure provide an apparatus and method for detecting a living body and a system, which may efficiently eliminate influences of other stationary objects and noises, and achieve accurate detection of a position of a living body.

According to a first aspect of the embodiments of this disclosure, there is provided an apparatus for detecting a living body, including: a first calculating unit configured to obtain range FFT signals within a first preset time range according to reflected signals of a microwave radar within the first preset time range; a second calculating unit configured to obtain amplitude distributions and/or phase distributions of distances in the first preset time range according to the range FFT signals within the first preset time range, and calculate amplitude fluctuations of the distances within the first preset time range; a third calculating unit configured to perform Fourier transform on the amplitude distributions and/or the phase distributions to obtain amplitude spectra and/or phase spectra; and a first determining unit configured to, based on magnitudes of the amplitude fluctuations, determine whether there exist living bodies at the distances according to the amplitude spectra and/or the phase spectra and the amplitude fluctuations, or determine whether there exist living bodies at the distances according to the amplitude spectra and/or the phase spectra.

According to a second aspect of the embodiments of this disclosure, there is provided a system for detecting a living body, including: a microwave radar including a signal transmission portion and a signal reception portion, the signal transmission portion transmitting microwave signals to a space where the living body is located, and the signal reception portion receiving reflected signals; and the apparatus for detecting a living body as described in the first aspect of the embodiments of this disclosure, which performs living body detection according to the reflected signals.

According to a third aspect of the embodiments of this disclosure, there is provided a method for detecting a living body, including: obtaining range FFT signals within a first preset time range according to reflected signals of a microwave radar within the first preset time range; obtaining amplitude distributions and/or phase distributions of distances in the first preset time range according to the range FFT signals within the first preset time range, and calculating amplitude fluctuations of the distances within the first preset time range; performing Fourier transform on the amplitude distributions and/or the phase distributions to obtain amplitude spectra and/or phase spectra; and based on magnitudes of the amplitude fluctuations, determining whether there exist living bodies at the distances according to the amplitude spectra and/or the phase spectra and the amplitude fluctuations, or determining whether there exist living bodies at the distances according to the amplitude spectra and/or the phase spectra.

An advantage of the embodiments of this disclosure exists in that as an obvious amplitude fluctuation may reflect a micromovement of a living body and the amplitude spectrum and/or phase spectrum may reflect frequency distribution caused by regular micromovements of the living body, based on magnitudes of amplitude fluctuations of range FFT signals at various distances within a certain period of time, whether the amplitude fluctuations are taken into account in detecting the living body is determined, and whether there exist living bodies at the distances is determined with reference to amplitude spectra and/or phase spectra of the range FFT signals, thereby efficiently eliminating influences of other stationary objects and noises, and achieving accuracy detection of the position of the living body.

With reference to the following description and drawings, the particular embodiments of this disclosure are disclosed in detail, and the principle of this disclosure and the manners of use are indicated. It should be understood that the scope of the embodiments of this disclosure is not limited thereto. The embodiments of this disclosure contain many alternations, modifications and equivalents within the scope of the terms of the appended claims.

Features that are described and/or illustrated with respect to one embodiment may be used in the same way or in a similar way in one or more other embodiments and/or in combination with or instead of the features of the other embodiments.

It should be emphasized that the term “comprises/comprising/includes/including” when used in this specification is taken to specify the presence of stated features, integers, steps or components but does not preclude the presence or addition of one or more other features, integers, steps, components or groups thereof.

### BRIEF DESCRIPTION OF THE DRAWINGS

The drawings are included to provide further understanding of this disclosure, which constitute a part of the specification and illustrate the preferred embodiments of this disclosure, and are used for setting forth the principles of this disclosure together with the description. It is obvious that the accompanying drawings in the following description are some embodiments of this disclosure, and for those of ordinary skills in the art, other accompanying drawings may be obtained according to these accompanying drawings without making an inventive effort. In the drawings:

FIG. 1 is a schematic diagram of the apparatus for detecting a living body of Embodiment 1 of this disclosure;

FIG. 2 is a schematic diagram of transmitting and receiving signals by a microwave radar of Embodiment 1 of this disclosure;

FIG. 3 is a schematic diagram of a range FFT signal of Embodiment 1 of this disclosure;

FIG. 4 is a schematic diagram of amplitude distribution of a first distance of Embodiment 1 of this disclosure;

FIG. 5 is a schematic diagram of an amplitude spectrum of the first distance of Embodiment 1 of this disclosure;

FIG. 6 is a schematic diagram of amplitude distribution of a second distance of Embodiment 1 of this disclosure;

FIG. 7 is a schematic diagram of an amplitude spectrum of the second distance of Embodiment 1 of this disclosure;

FIG. 8 is a schematic diagram of phase distribution of the first distance of Embodiment 1 of this disclosure;

FIG. 9 is a schematic diagram of a phase spectrum of the first distance of Embodiment 1 of this disclosure;

FIG. 10 is a schematic diagram of phase distribution of the second distance of Embodiment 1 of this disclosure;

FIG. 11 is a schematic diagram of a phase spectrum of the second distance of Embodiment 1 of this disclosure;

FIG. 12 is a schematic diagram of a first determining unit 104 of Embodiment 1 of this disclosure;

FIG. 13 is a schematic diagram of a second determining unit 1201 of Embodiment 1 of this disclosure;

FIG. 14 is a schematic diagram of a method for determining whether a living body exists at a certain distance by the fourth determining unit 1302 of Embodiment 1 of this disclosure;

FIG. 15 is a schematic diagram of a third determining unit 1202 of Embodiment 1 of this disclosure;

FIG. 16 is a schematic diagram of a method for determining whether a living body exists at a certain distance by the fifth determining unit 1502 of Embodiment 1 of this disclosure;

FIG. 17 is a schematic diagram of the electronic device of Embodiment 2 of this disclosure;

FIG. 18 is a block diagram of a system structure of the electronic device of Embodiment 2 of this disclosure;

FIG. 19 is a schematic diagram of the system for detecting a living body of Embodiment 3 of this disclosure;

FIG. 20 is a schematic diagram of the method for detecting a living body of Embodiment 4 of this disclosure.

### DETAILED DESCRIPTION

These and further aspects and features of this disclosure will be apparent with reference to the following description and attached drawings. In the description and drawings, particular embodiments of the disclosure have been disclosed in detail as being indicative of some of the ways in which the principles of the disclosure may be employed, but it is understood that the disclosure is not limited correspondingly in scope. Rather, the disclosure includes all changes, modifications and equivalents coming within the spirit terms of the appended claims. Various embodiments of this disclosure shall be described below with reference to the accompanying drawings, and these embodiments are illustrative only, and are not intended to limit this disclosure.

### Embodiment 1

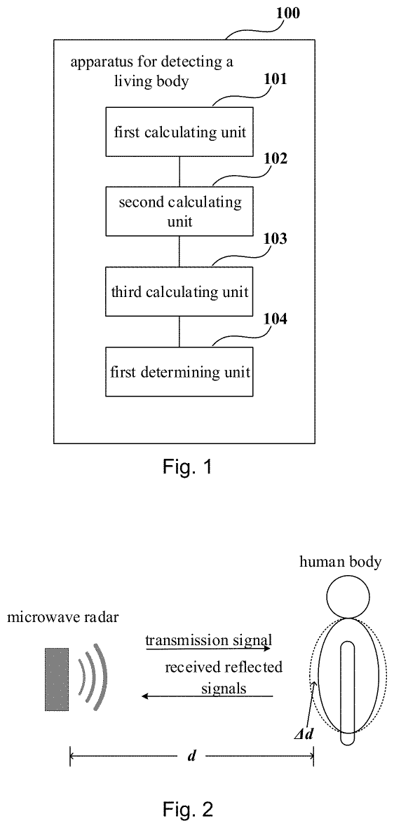

This embodiment of this disclosure provides an apparatus for detecting a living body. FIG. 1 is a schematic diagram of the apparatus for detecting a living body of Embodiment 1 of this disclosure. As shown in FIG. 1, an apparatus 100 for detecting a living body includes: a first calculating unit 101 configured to obtain range FFT signals within a first preset time range according to reflected signals of a microwave radar within the first preset time range; a second calculating unit 102 configured to obtain amplitude distributions and/or phase distributions of distances in the first preset time range according to the range FFT signals within the first preset time range, and calculate amplitude fluctuations of the distances within the first preset time range; a third calculating unit 103 configured to perform Fourier transform on the amplitude distributions and/or the phase distributions to obtain amplitude spectra and/or phase spectra; and a first determining unit 104 configured to, based on magnitudes of the amplitude fluctuations, determine whether there exist living bodies at the distances according to the amplitude spectra and/or the phase spectra and the amplitude fluctuations, or determine whether there exist living bodies at the distances according to the amplitude spectra and/or the phase spectra.

Therefore, as an obvious amplitude fluctuation may reflect a micromovement of a living body and the amplitude spectrum and/or phase spectrum may reflect frequency distribution caused by regular micromovements of the living body, based on magnitudes of amplitude fluctuations of range FFT signals at various distances within a certain period of time, whether the amplitude fluctuations are taken into account in detecting the living body is determined, and whether there exist living bodies at the distances is determined with reference to amplitude spectra and/or phase spectra of the range FFT signals, thereby efficiently eliminating influences of other stationary objects and noises, and achieving accuracy detection of the position of the living body.

In this embodiment, the apparatus for detecting a living body may be used for detection of various living bodies. In this example, description shall be given by taking a human body as a detected object as an example.

The first calculating unit 101 obtains range fast Fourier transform (FFT) signals within a first preset time range according reflection signals of the microwave radar within the first preset time range.

In this embodiment, the micromovements of the living body are mainly caused by breathing. Therefore, the first preset time range at least includes one breathing cycle, and a particular range thereof may be set as actually demanded.

For example, taking a human body as an example, a breathing cycle may be 3-6 seconds, and a heartbeat cycle may be 0.3-1.2 seconds.

In this embodiment, the microwave radar may be one with an operational mode being frequency-modulated continuous waves (FMCWs).

FIG. 2 is a schematic diagram of transmitting and receiving signals by the microwave radar of Embodiment 1 of this disclosure. As shown in FIG. 2, a transmission signal of the microwave radar is reflected by objects including the human body and then received by the microwave radar. The microwave radar processes the transmission signal and the received reflected signals to obtain a difference frequency signal. The signal received by the microwave radar is a superposition of all reflected signals in space. The signal may be decomposed by performing fast Fourier transform on the difference frequency signal so as to obtain reflected signals at different distances. This Fourier transform is referred to as range FFT. The range FFT signal obtained after the range FFT processing may be expressed by formula (1) below:

S=Asin(2πft+p) (1);

where, A is an amplitude, p is a phase, a frequency f is subjected to a distance between the human body and the radar, f=s2d/c, s being a slope of microwave radar transmission signal frequency modulation, d being the distance between the human body and the radar, and c being the speed of light.

It can be seen that the amplitude A of the range FFT signal is subjected to such factors as the distance d from the human body to the radar, a characteristic of a reflecting surface of the human body, and a micromovement of the human body, and the phase p of the range FFT signal is mainly subjected to the micromovement Δd of the human body.

In this embodiment, for each distance from the radar, a range FFT signal of the distance may be obtained. In some cases, due to limitations of such factors as signal processing, and sampling resolution, distance points able to obtain range FFT signals are not continuous, but are multiple discrete distance points, which are also referred to as range bins. However, the embodiments of this disclosure are not limited to these discrete range bins, and they may all continuous distance points.

In this embodiment, the second calculation unit 102 obtains the amplitude distributions and/or the phase distributions of the distances in the first preset time range (t₁, tₙ) according to the range FFT signals within the first preset time range, the amplitude distributions being able to be denoted by curves of amplitudes along with the time, and the phase distributions being able to be denoted by curves of phases along with the time, and the second calculation unit 102 calculates amplitude fluctuations of the distances within the first preset time range.

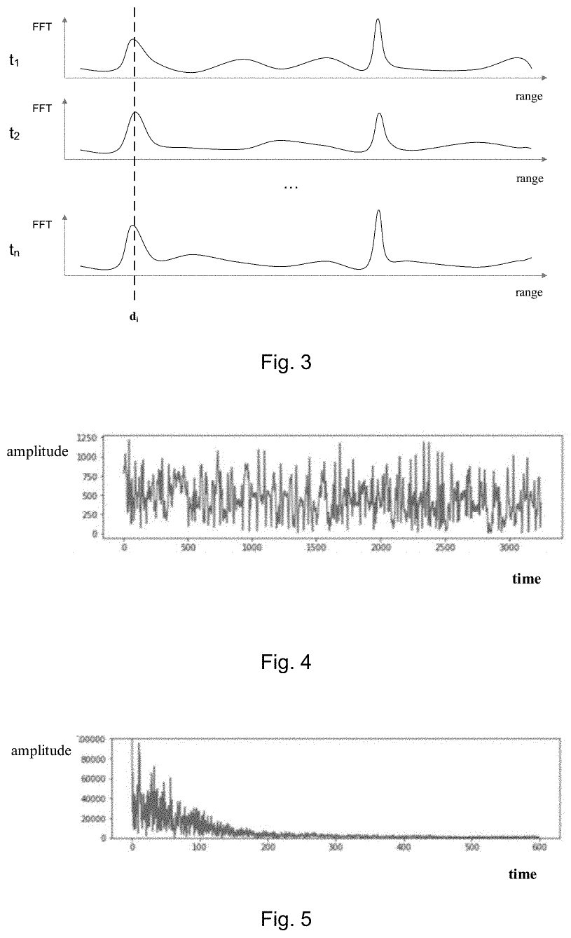

FIG. 3 is a schematic diagram of the range FFT signal of Embodiment 1 of this disclosure. As shown in FIG. 3, the abscissa is the distance from the radar, and the ordinate is the amplitude or phase of the range FFT signal. From top to bottom, they are amplitude information or phase information of the range FFT signal at time t₁, time t₂, . . . , tₙ.

As shown in FIG. 3, for a certain distance dᵢ, a change in its amplitude or phase from time t₁ to time tₙ reflects the amplitude or phase distribution within the first preset time range.

For example, for the distance dᵢ, variance of the amplitude from time t₁ to time tₙ may be used to measure the amplitude fluctuation.

In this embodiment, the third calculation unit 103 performs Fourier transform on the amplitude distributions and/or the phase distributions to obtain the amplitude spectra and/or phase spectra, the amplitude spectra being able to be denoted by curves of amplitudes along with the time, and the phase spectra being able to be denoted by curves of phases along with the time, and the Fourier transform being, for example, fast Fourier transform (FFT).

With the second calculating unit 102 and the third calculating unit 103, the amplitude fluctuations, amplitude distributions and/or phase distributions, and amplitude spectra and/or phase spectra of the distances may be obtained.

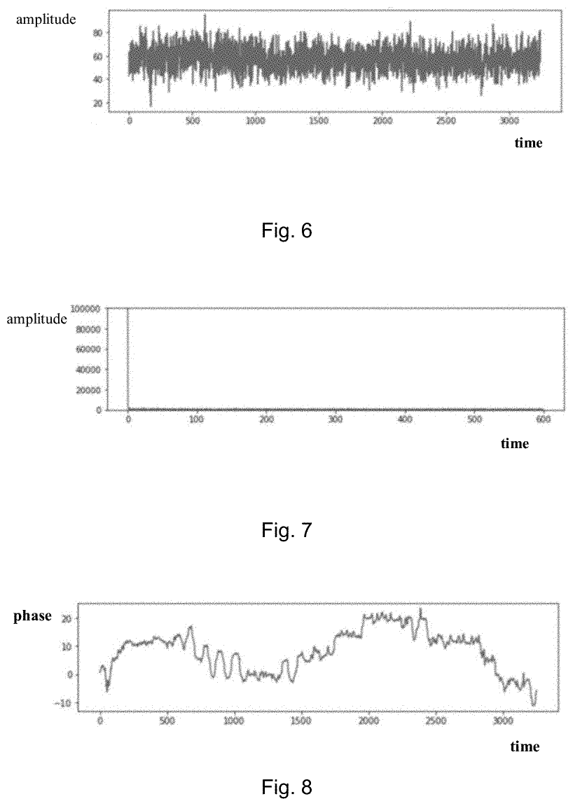

FIG. 4 is a schematic diagram of amplitude distribution of a first distance of Embodiment 1 of this disclosure, FIG. 5 is a schematic diagram of an amplitude spectrum of the first distance of Embodiment 1 of this disclosure, FIG. 6 is a schematic diagram of amplitude distribution of a second distance of Embodiment 1 of this disclosure, and FIG. 7 is a schematic diagram of an amplitude spectrum of the second distance of Embodiment 1 of this disclosure.

As shown in FIG. 5 and FIG. 7, the distribution of the amplitude spectrum of the first distance is significantly different from the distribution of the amplitude spectrum of the second distance.

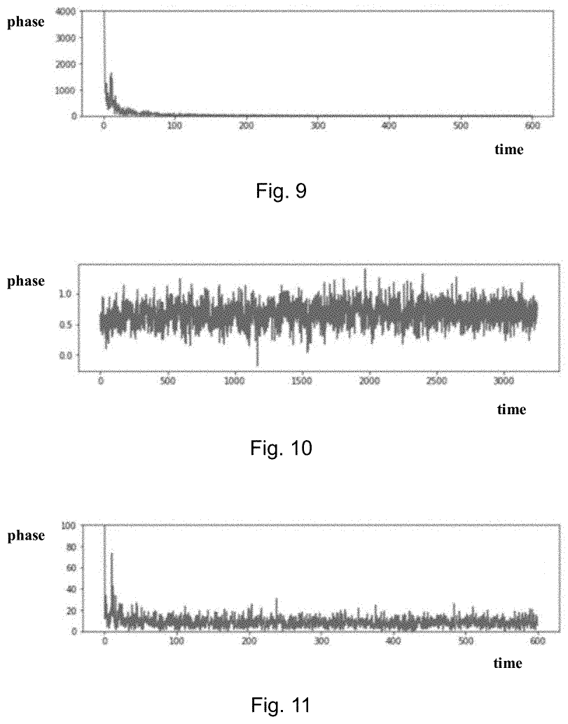

FIG. 8 is a schematic diagram of phase distribution of the first distance of Embodiment 1 of this disclosure, FIG. 9 is a schematic diagram of a phase spectrum of the first distance of Embodiment 1 of this disclosure, FIG. 10 is a schematic diagram of phase distribution of the second distance of Embodiment 1 of this disclosure, and FIG. 11 is a schematic diagram of a phase spectrum of the second distance of Embodiment 1 of this disclosure.

As shown in FIG. 9 and FIG. 11, the distribution of the phase spectrum of the first distance is also relatively significantly different from the distribution of the phase spectrum of the second distance.

After obtaining the amplitude fluctuations, the amplitude distributions and/or the phase distributions, the amplitude spectra and/or phase spectra of the distances, based on magnitudes of the amplitude fluctuations, the first determining unit 104 determines whether living bodies exists at the distances according to the amplitude spectra and/or the phase spectra and the amplitude fluctuations, or determines whether living bodies exists at the distances according to the amplitude spectra and/or the phase spectra.

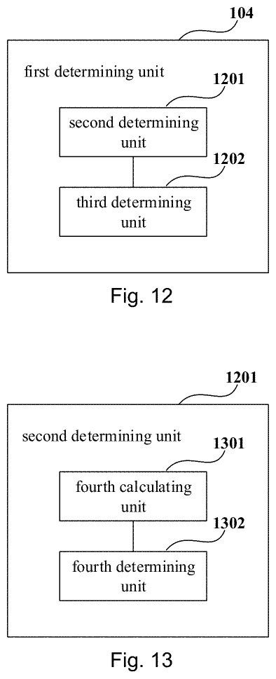

FIG. 12 is a schematic diagram of the first determining unit 104 of Embodiment 1 of this disclosure. As shown in FIG. 12, the first determining unit 104 includes: a second determining unit 1201 configured to, when the amplitude fluctuations are greater than a first threshold, determine whether there exist living bodies at the distances according to the amplitude spectra and/or the phase spectra and the amplitude fluctuations; and a third determining unit 1202 configured to, when the amplitude fluctuations are less than or equal to the first threshold, determine whether there exist living bodies at the distances according to the amplitude spectra and/or the phase spectra.

In this embodiment, the first threshold may be set as actually demanded.

In this embodiment, according to a relationship between an amplitude fluctuation of a certain distance and the first threshold, whether the second determining unit 1201 or the third determining unit 1202 determines that there exists a living body at the distance may be determined.

In this embodiment, the second determining unit 1201 determines whether there exist living bodies at the distances according to first distribution of the amplitude spectra the distances on a frequency and/or second distribution of the phase spectra of the distances on the frequency and the amplitude fluctuations of the distances.

A method for determining whether there exist living bodies at the distances by the second determining unit 1201 shall be particularly described below.

FIG. 13 is a schematic diagram of the second determining unit 1201 of Embodiment 1 of this disclosure. As shown in FIG. 13, the second determining unit 1201 includes: a fourth calculating unit 1301 configured to calculate amplitude low-frequency energy ratios and/or phase low-frequency energy ratios of the distances according to the first distributions of the amplitude spectra of the distances in a frequency and/or the second distributions of the phase spectra of the distances in the frequency; and a fourth determining unit 1302 configured to determine that there exists a living body on a distance when amplitude fluctuations of all distances in a local range centered at the distance are greater than the first threshold and at least one of the following conditions is satisfied: amplitude low-frequency energy ratios of all the distances in the local range centered at the distance are all greater than a second threshold; and phase low-frequency energy ratios of all the distances in the local range centered at the distance are all greater than a third threshold.

In this embodiment, the fourth calculating unit 1301 calculates the amplitude low-frequency energy ratios and/or phase low-frequency energy ratios of the distances according to the first distribution of the amplitude spectra of the distances in the frequency and/or the second distribution of the phase spectra of the distances in the frequency.

For example, ratios of energies of the amplitudes and/or phases in the preset frequency range related to the living bodies to energies of amplitudes and/or phases in a non-negative frequency range at the distances are calculated to obtain the amplitude low-frequency energy ratios and/or phase low-frequency energy ratios of the distances.

In this embodiment, the preset frequency range related to the living bodies is, for example, a range determined according to a frequency of breathing. The frequency of breathing is a low frequency part in comparison to a frequency of a noise.

For example, the amplitude low frequency energy ratio and/or the phase low frequency energy ratio may be calculated according to formula (2) below:

$$r = \frac{{Σ}_{i ∈ ( {f}_{1} , {f}_{2} )} {e}_{i}^{2}}{{Σ}_{j ∈ ( {f}_{1} , F )} {e}_{j}^{2}} ; \qquad ( 2 )$$

where, r denotes the amplitude low-frequency energy ratio or the phase low-frequency energy ratio, e denotes the amplitude of the amplitude spectrum or the phase spectrum, (f₁,f₂) is a normal breathing frequency range, and F is a highest frequency of the amplitude spectrum or the phase spectrum.

For another example, high-pass filtering may be performed on the amplitude spectrum or the phase spectrum to obtain a filtering result D_{H}=(d₁^{H}, d₂^{H}, . . . , d_{T}^{H}) with a frequency greater than f₁, and band-pass filtering may be performed on the amplitude spectrum or the phase spectrum to obtain a filtering result D_{B}=(d₁^{B}, d₂^{B}, . . . , d_{T}^{B}) with a frequency between (f₁, f₂), the amplitude low frequency energy ratio and/or phase low frequency energy ratio being a ratio of an energy of D_{H} to an energy of D_{B}. For example, the amplitude low-frequency energy ratio and/or the phase low-frequency energy ratio may be calculated according to the formula (3) below:

$$r = \frac{{Σ}_{i ∈ {D}_{B}} {d}_{i}^{2}}{{Σ}_{j ∈ {D}_{H}} {d}_{j}^{2}} ; \qquad ( 3 )$$

where, r denotes the amplitude low-frequency energy ratio or the phase low-frequency energy ratio, D_{H} denotes the result of high-pass filtering, and D_{B} denotes the result of band-pass filtering.

In determining whether there exist living bodies at the distances, the fourth determining unit 1302 may determine one by one according to the distances.

For a certain distance therein, when amplitude fluctuations of all distances in a local range centered at the distance are greater than the first threshold and when amplitude low-frequency energy ratios of all the distances in the local range centered at the distance are all greater than the second threshold and/or the phase low-frequency energy ratios of all the distances in the local range centered at the distance are all greater than the third threshold, it is determined that there exists a living body at the distance.

For example, when an amplitude fluctuation of the distance is of a local maximum value in the local range centered at the distance, the amplitude fluctuations of all the distances in the local range centered at the distance are greater than the first threshold, the amplitude low-frequency energy ratios of all the distances in the local range centered at the distance are all greater than the second threshold and the phase low-frequency energy ratios of all the distances in the local range centered at the distance are all greater than the third threshold, the fourth determining unit 1302 determines that there exists a living body at the distance.

In this embodiment, a size of the local range, the second threshold and the third threshold may be set as actually demanded.

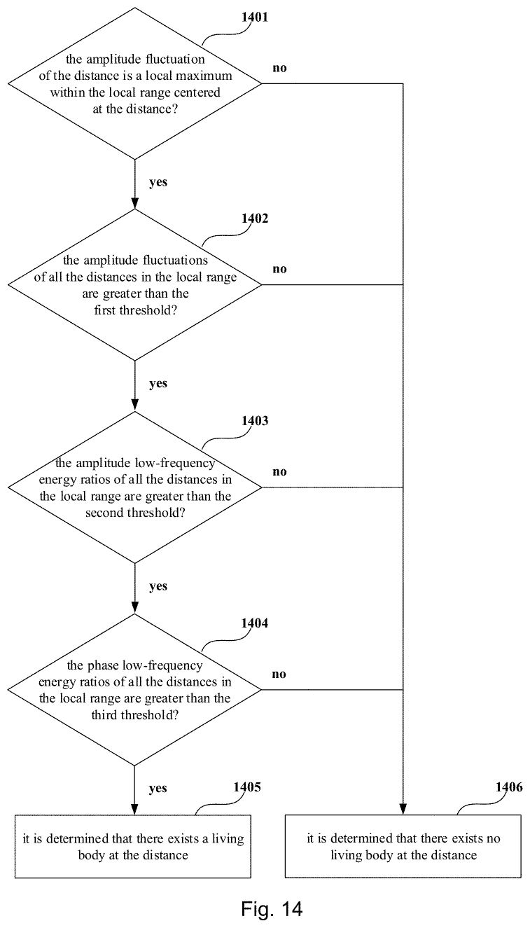

FIG. 14 is a schematic diagram of a method for determining whether a living body exists at a certain distance by the fourth determining unit 1302 of Embodiment 1 of this disclosure. As shown in FIG. 14, the method includes:

Step 1401: it is determined whether the amplitude fluctuation of the distance is a local maximum within the local range centered at the distance, entering into step 1402 when it is determined “yes”, and entering into step 1406 when it is determined “no”;

Step 1402: it is determined whether the amplitude fluctuations of all the distances in the local range are greater than the first threshold, entering into step 1403 when it is determined “yes”, and entering into step 1406 when it is determined “no”;

Step 1403: it is determined whether the amplitude low-frequency energy ratios of all the distances in the local range are greater than the second threshold, entering into step 1404 when it is determined “yes”, and entering into step 1406 when it is determined “no”;

Step 1404: it is determined whether the phase low-frequency energy ratios of all the distances in the local range are greater than the third threshold, entering into step 1405 when it is determined “yes”, and entering into step 1406 when it is determined “no”;

Step 1405: it is determined that there exists a living body at the distance;

Step 1406: it is determined that there exists no living body at the distance.

The above steps are repeatedly performed on all the distance so as to obtain detection results of whether there exist living bodies at all the distances.

The method for determining whether there exist living bodies at all the distances by the second determining unit 1201 is described above.

A method for determining whether there exist living bodies at all the distances by the third determining unit 1202 shall be described below.

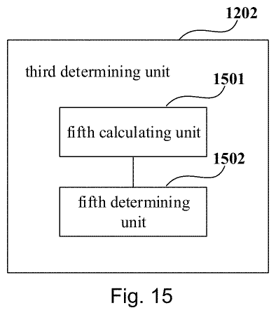

FIG. 15 is a schematic diagram of the third determining unit 1202 of Embodiment 1 of this disclosure. As shown in FIG. 15, the third determining unit 1202 includes: a fifth calculating unit 1501 configured to calculate the amplitude low-frequency energy ratios and/or the phase low-frequency energy ratios of the distances according to the first distribution of the amplitude spectra of the distances in a frequency and/or the second distribution of the phase spectra of the distances in a frequency; and a fifth determining unit 1502 configured to determine that there exists a living body at the distance when at least one of the following conditions is satisfied: the amplitude low-frequency energy ratios of all the distances in a local range centered at the distance are all greater than the second threshold; and the phase low-frequency energy ratios of all the distances in the local range centered at the distance are all greater than the third threshold.

In this embodiment, a calculation method of the fifth calculating unit 1501 is identical to that of the fourth calculating unit 1301, and shall not be described herein any further.

In this embodiment, when the amplitude low-frequency energy ratios of all the distances in the local range centered at the distance are all greater than the second threshold and/or the phase low-frequency energy ratios of all the distances in the local range centered at the distance are all greater than the third threshold, the fifth determining unit 1502 determines that there exists a living body at the distance.

For example, when the amplitude low-frequency energy ratio of the distance is of a local maximum value in the local range centered at the distance, the amplitude low-frequency energy ratios of all the distances in the local range centered at the distance are all greater than the second threshold and the phase the low-frequency energy ratios of all the distances in the local range centered at the distance are all greater than the third threshold, the fifth determining unit determines that there exists a living body at the distance.

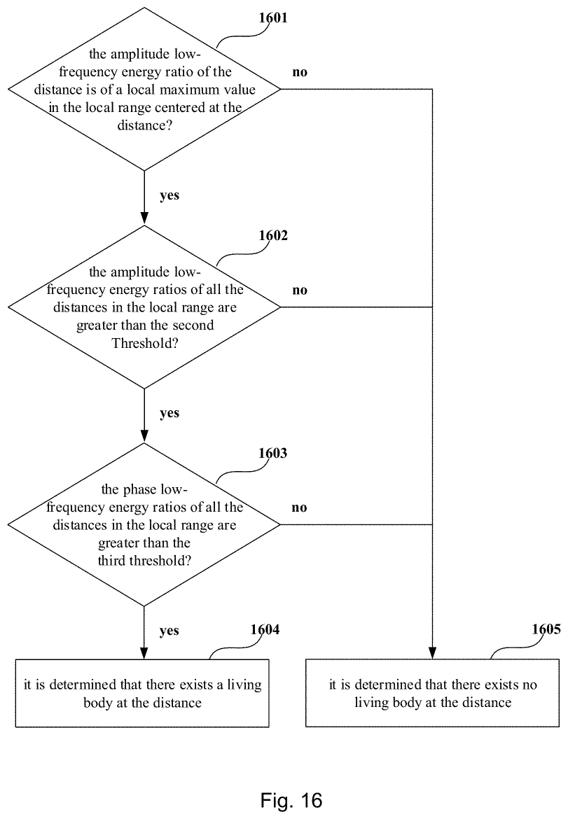

FIG. 16 is a schematic diagram of a method for determining whether a living body exists at a certain distance by the fifth determining unit 1502 of Embodiment 1 of this disclosure. As shown in FIG. 16, the method includes:

Step 1601: it is determined whether the amplitude low-frequency energy ratio of the distance is of a local maximum value in the local range centered at the distance, entering into step 1602 when it is determined “yes”, and entering into step 1605 when it is determined “no”;

Step 1602: it is determined whether the amplitude low-frequency energy ratios of all the distances in the local range are greater than the second threshold, entering into step 1603 when it is determined “yes”, and entering into step 1605 when it is determined “no”;

Step 1603: it is determined whether the phase low-frequency energy ratios of all the distances in the local range are greater than the third threshold, entering into step 1604 when it is determined “yes”, and entering into step 1605 when it is determined “no”;

Step 1604: it is determined that there exists a living body at the distance;

Step 1605: it is determined that there exists no living body at the distance.

The above steps are repeatedly performed on all the distance so as to obtain detection results of whether there exist living bodies at all the distances.

As shown in FIG. 5 and FIG. 9, the distribution of the amplitude spectrum and phase spectrum of the first distance are concentrated at a low-frequency portion, and with the above particular judgment steps, it is determined that there exists a living body at the first distance.

As shown in FIG. 7 and FIG. 11, the distribution of the amplitude spectrum and phase spectrum of the second distance are not concentrated at a low-frequency portion, and with the above particular judgment steps, it is determined that there exists no living body at the first distance.

It can be seen from the above embodiment that as an obvious amplitude fluctuation may reflect a micromovement of a living body and the amplitude spectrum and/or phase spectrum may reflect frequency distribution caused by regular micromovements of the living body, based on magnitudes of amplitude fluctuations of range FFT signals at various distances within a certain period of time, whether the amplitude fluctuations are taken into account in detecting the living body is determined, and whether there exist living bodies at the distances is determined with reference to amplitude spectra and/or phase spectra of the range FFT signals, thereby efficiently eliminating influences of other stationary objects and noises, and achieving accuracy detection of the position of the living body.

### Embodiment 2

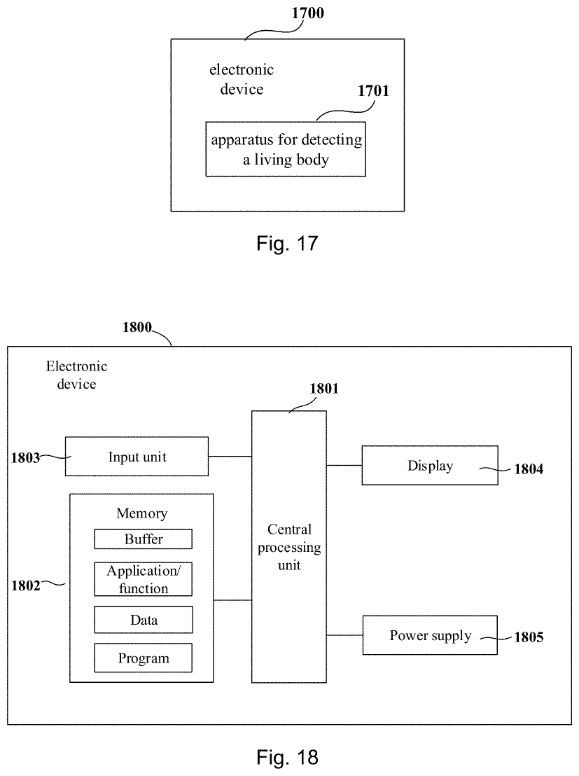

This embodiment of this disclosure provides an electronic device. FIG. 17 is a schematic diagram of the electronic device of Embodiment 2 of this disclosure. As shown in FIG. 17, an electronic device 1700 includes an apparatus 1701 for detecting a living body, a structure and functions of which being identical to those contained in Embodiment 1, and being not going to be described herein any further.

FIG. 18 is a block diagram of a systematic structure of the electronic device of Embodiment 2 of this disclosure. As shown in FIG. 18, an electronic device 1800 may include a central processing unit 1801 and a memory 1802, the memory 1802 being coupled to the central processing unit 1801. This figure is illustrative only, and other types of structures may also be used, so as to supplement or replace this structure and achieve a telecommunications function or other functions.

As shown in FIG. 18, the electronic device 1800 may include an input unit 1803, a display 1804, and a power supply 1805.

For example, the functions of the apparatus for detecting a living body described in Embodiment 1 may be integrated into the central processing unit 1801. For example, the central processing unit 1801 may be configured to: obtain range FFT signals within a first preset time range according to reflected signals of a microwave radar within the first preset time range; obtain amplitude distributions and/or phase distributions of distances in the first preset time range according to the range FFT signals within the first preset time range, and calculate amplitude fluctuations of the distances within the first preset time range; perform Fourier transform on the amplitude distributions and/or the phase distributions to obtain amplitude spectra and/or phase spectra; and based on magnitudes of the amplitude fluctuations, determine whether there exist living bodies at the distances according to the amplitude spectra and/or the phase spectra and the amplitude fluctuations, or determine whether there exist living bodies at the distances according to the amplitude spectra and/or the phase spectra.

For example, based on magnitudes of the amplitude fluctuations, determining whether there exist living bodies at the distances according to the amplitude spectra and/or the phase spectra and the amplitude fluctuations, or determining whether there exist living bodies at the distances according to the amplitude spectra and/or the phase spectra, includes: when the amplitude fluctuations are greater than a first threshold, determining whether there exist living bodies at the distances according to the amplitude spectra and/or the phase spectra and the amplitude fluctuations, and when the amplitude fluctuations are less than or equal to the first threshold, determining whether there exist living bodies at the distances according to the amplitude spectra and/or the phase spectra.

For example, the determining whether there exist living bodies at the distances according to the amplitude spectra and/or the phase spectra and the amplitude fluctuations includes: determining whether there exist living bodies at the distances according to first distributions of the amplitude spectra of the distances in a frequency and/or second distributions of the phase spectra of the distances in a frequency and the amplitude fluctuations of the distances, and the determining whether there exist living bodies at the distances according to the amplitude spectra and/or the phase spectra includes: determining whether there exist living bodies at the distances according to the first distributions of the amplitude spectra of the distances in a frequency and/or the second distributions of the phase spectra of the distances in a frequency.

For example, the determining whether there exist living bodies at the distances according to first distributions of the amplitude spectra of the distances in a frequency and/or second distributions of the phase spectra of the distances in a frequency and the amplitude fluctuations of the distances includes: calculating amplitude low-frequency energy ratios and/or phase low-frequency energy ratios of the distances according to the first distributions of the amplitude spectra of the distances in a frequency and/or the second distributions of the phase spectra of the distances in the frequency, and determining that there exists a living body on a distance when amplitude fluctuations of all distances in a local range centered at the distance are greater than the first threshold and at least one of the following conditions is satisfied: amplitude low-frequency energy ratios of all the distances in the local range centered at the distance are all greater than a second threshold; and phase low-frequency energy ratios of all the distances in the local range centered at the distance are all greater than a third threshold.

For example, when an amplitude fluctuation of the distance is of a local maximum value in the local range centered at the distance, the amplitude fluctuations of all the distances in the local range centered at the distance are greater than the first threshold, the amplitude low-frequency energy ratios of all the distances in the local range centered at the distance are all greater than the second threshold, and phase the low-frequency energy ratios of all the distances in the local range centered at the distance are all greater than the third threshold, it is determined that there exists a living body at the distance.

For example, the determining whether there exist living bodies at the distances according to first distributions of the amplitude spectra of the distances in a frequency and/or second distributions of the phase spectra of the distances in a frequency includes: calculating amplitude low-frequency energy ratios and/or phase low-frequency energy ratios of the distances according to the first distributions of the amplitude spectra of the distances in a frequency and/or the second distributions of the phase spectra of the distances in the frequency, and determining that there exists a living body at the distance when at least one of the following conditions is satisfied: amplitude low-frequency energy ratios of all the distances in the local range centered at the distance are all greater than a second threshold; and phase low-frequency energy ratios of all the distances in the local range centered at the distance are all greater than a third threshold.

For example, when an amplitude low-frequency energy ratio of the distance is of a local maximum value in the local range centered at the distance, the amplitude low-frequency energy ratios of all the distances in the local range centered at the distance are all greater than the second threshold, and the phase the low-frequency energy ratios of all the distances in the local range centered at the distance are all greater than the third threshold, it is determined that there exists a living body at the distance.

For another example, the apparatus for detecting a living body described in Embodiment 1 and the central processing unit 1801 may be configured separately. For example the apparatus for detecting a living body may be configured as a chip connected to the central processing unit 1801, and the functions of the apparatus for detecting a living body are executed under control of the central processing unit 1801.

In this embodiment, the electronic device 1800 does not necessarily include all the components shown in FIG. 18.

As shown in FIG. 18, the central processing unit 1801 is sometimes referred to as a controller or control, which may include a microprocessor or other processor devices and/or logic devices, and the central processing unit 1801 receives input and controls operations of every component of the electronic device 1800.

The memory 1802 may be, for example, one or more of a buffer memory, a flash memory, a hard drive, a mobile medium, a volatile memory, a nonvolatile memory, or other suitable devices, which may store the information on configuration, etc., and furthermore, store programs executing related information. And the central processing unit 1801 may execute programs stored in the memory 1802, so as to realize information storage or processing, etc. Functions of other parts are similar to those of the related art, which shall not be described herein any further. The parts of the terminal device, or the electronic device 1800 may be realized by specific hardware, firmware, software, or any combination thereof, without departing from the scope of this disclosure.

It can be seen from the above embodiment that as an obvious amplitude fluctuation may reflect a micromovement of a living body and the amplitude spectrum and/or phase spectrum may reflect frequency distribution caused by regular micromovements of the living body, based on magnitudes of amplitude fluctuations of range FFT signals at various distances within a certain period of time, whether the amplitude fluctuations are taken into account in detecting the living body is determined, and whether there exist living bodies at the distances is determined with reference to amplitude spectra and/or phase spectra of the range FFT signals, thereby efficiently eliminating influences of other stationary objects and noises, and achieving accuracy detection of the position of the living body.

### Embodiment 3

This embodiment of this disclosure provides a system for detecting a living body, including a microwave radar and an apparatus for detecting a living body, a structure and functions of the apparatus for detecting a living body being identical those contained in Embodiment 1, and being not going to be described herein any further.

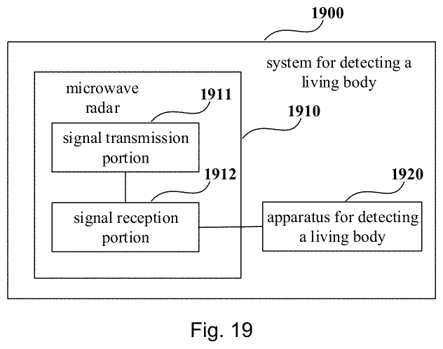

FIG. 19 is a schematic diagram of the system for detecting a living body of Embodiment 3 of this disclosure. As shown in FIG. 19, a system 1900 for detecting a living body includes: a microwave radar 1910 including a signal transmission portion 1911 and a signal reception portion 1912, the signal transmission portion 1911 transmitting microwave signals to a space where the living body is located, and the signal reception portion 1912 receiving reflected signals; and an apparatus 1920 for detecting a living body, which performs living body detection according to the reflected signals.

For example, the microwave radar 1910 is one having a three-dimensional antenna array. Reference may be made related techniques for the signal transmission portion 1911 and the signal reception portion 1912 of the microwave radar 1910.

In this embodiment, a structure and functions of the apparatus 1920 for detecting a living body are identical to those contained in Embodiment 1, and shall not be described herein any further.

It can be seen from the above embodiment that as an obvious amplitude fluctuation may reflect a micromovement of a living body and the amplitude spectrum and/or phase spectrum may reflect frequency distribution caused by regular micromovements of the living body, based on magnitudes of amplitude fluctuations of range FFT signals at various distances within a certain period of time, whether the amplitude fluctuations are taken into account in detecting the living body is determined, and whether there exist living bodies at the distances is determined with reference to amplitude spectra and/or phase spectra of the range FFT signals, thereby efficiently eliminating influences of other stationary objects and noises, and achieving accuracy detection of the position of the living body.

### Embodiment 4

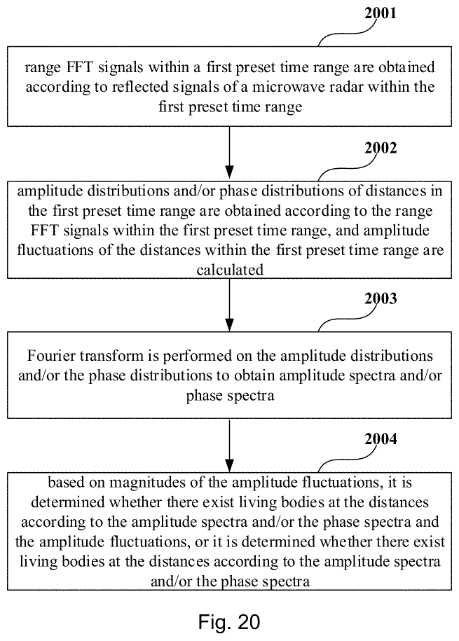

This embodiment of this disclosure provides a method for detecting a living body, corresponding to the apparatus for detecting a living body of Embodiment 1. FIG. 20 is a schematic diagram of the method for detecting a living body of Embodiment 4 of this disclosure. As shown in FIG. 20, the method includes:

Step 2001: range FFT signals within a first preset time range are obtained according to reflected signals of a microwave radar within the first preset time range;

Step 2002: amplitude distributions and/or phase distributions of distances in the first preset time range are obtained according to the range FFT signals within the first preset time range, and amplitude fluctuations of the distances within the first preset time range are calculated;

Step 2003: Fourier transform is performed on the amplitude distributions and/or the phase distributions to obtain amplitude spectra and/or phase spectra; and

Step 2004: based on magnitudes of the amplitude fluctuations, it is determined whether there exist living bodies at the distances according to the amplitude spectra and/or the phase spectra and the amplitude fluctuations, or it is determined whether there exist living bodies at the distances according to the amplitude spectra and/or the phase spectra.

In this embodiment, particular implementations of the above steps are identical to those contained in Embodiment 1, and shall not be described herein any further.

It can be seen from the above embodiment that as an obvious amplitude fluctuation may reflect a micromovement of a living body and the amplitude spectrum and/or phase spectrum may reflect frequency distribution caused by regular micromovements of the living body, based on magnitudes of amplitude fluctuations of range FFT signals at various distances within a certain period of time, whether the amplitude fluctuations are taken into account in detecting the living body is determined, and whether there exist living bodies at the distances is determined with reference to amplitude spectra and/or phase spectra of the range FFT signals, thereby efficiently eliminating influences of other stationary objects and noises, and achieving accuracy detection of the position of the living body.

An embodiment of the present disclosure provides a computer readable program code, which, when executed in an apparatus for detecting a living body or an electronic device, will cause a computer to carry out the method for detecting a living body as described in Embodiment 4 in the apparatus for detecting a living body or the electronic device.

An embodiment of the present disclosure provides a computer storage medium, including a computer readable program, which will cause a computer to carry out the method for detecting a living body as described in Embodiment 4 in an apparatus for detecting a living body or an electronic device.

The method for detecting a living body carried out in the apparatus for detecting a living body or the electronic device as described with reference to the embodiments of this disclosure may be directly embodied as hardware, software modules executed by a processor, or a combination thereof. For example, one or more functional block diagrams and/or one or more combinations of the functional block diagrams shown in FIG. 1 may either correspond to software modules of procedures of a computer program, or correspond to hardware modules. Such software modules may respectively correspond to the steps shown in FIG. 16. And the hardware module, for example, may be carried out by firming the soft modules by using a field programmable gate array (FPGA).

The soft modules may be located in an RAM, a flash memory, an ROM, an EPROM, and EEPROM, a register, a hard disc, a floppy disc, a CD-ROM, or any memory medium in other forms known in the art. A memory medium may be coupled to a processor, so that the processor may be able to read information from the memory medium, and write information into the memory medium; or the memory medium may be a component of the processor. The processor and the memory medium may be located in an ASIC. The soft modules may be stored in a memory of a mobile terminal, and may also be stored in a memory card of a pluggable mobile terminal. For example, if equipment (such as a mobile terminal) employs an MEGA-SIM card of a relatively large capacity or a flash memory device of a large capacity, the soft modules may be stored in the MEGA-SIM card or the flash memory device of a large capacity.

One or more functional blocks and/or one or more combinations of the functional blocks in FIG. 1 may be realized as a universal processor, a digital signal processor (DSP), an application-specific integrated circuit (ASIC), a field programmable gate array (FPGA) or other programmable logic devices, discrete gate or transistor logic devices, discrete hardware component or any appropriate combinations thereof carrying out the functions described in this application. And the one or more functional block diagrams and/or one or more combinations of the functional block diagrams in FIG. 1 may also be realized as a combination of computing equipment, such as a combination of a DSP and a microprocessor, multiple processors, one or more microprocessors in communication combination with a DSP, or any other such configuration.

This disclosure is described above with reference to particular embodiments. However, it should be understood by those skilled in the art that such a description is illustrative only, and not intended to limit the protection scope of the present disclosure. Various variants and modifications may be made by those skilled in the art according to the principle of the present disclosure, and such variants and modifications fall within the scope of the present disclosure.

Following supplements are further disclosed in the embodiments of this disclosure:

A method for detecting a living body, may include obtaining range FFT signals within a first preset time range according to reflected signals of a microwave radar within the first preset time range; obtaining amplitude distributions and/or phase distributions of distances in the first preset time range according to the range FFT signals within the first preset time range, and calculating amplitude fluctuations of the distances within the first preset time range; performing Fourier transform on the amplitude distributions and/or the phase distributions to obtain amplitude spectra and/or phase spectra; and based on magnitudes of the amplitude fluctuations, determining whether there exist living bodies at the distances according to the amplitude spectra and/or the phase spectra and the amplitude fluctuations, or determining whether there exist living bodies at the distances according to the amplitude spectra and/or the phase spectra.

The based on magnitudes of the amplitude fluctuations, determining whether there exist living bodies at the distances according to the amplitude spectra and/or the phase spectra and the amplitude fluctuations, or determining whether there exist living bodies at the distances according to the amplitude spectra and/or the phase spectra, may include when the amplitude fluctuations are greater than a first threshold, determining whether there exist living bodies at the distances according to the amplitude spectra and/or the phase spectra and the amplitude fluctuations; and when the amplitude fluctuations are less than or equal to the first threshold, determining whether there exist living bodies at the distances according to the amplitude spectra and/or the phase spectra.

The determining whether there exist living bodies at the distances according to the amplitude spectra and/or the phase spectra and the amplitude fluctuations may include determining whether there exist living bodies at the distances according to first distributions of the amplitude spectra of the distances in a frequency and/or second distributions of the phase spectra of the distances in a frequency and the amplitude fluctuations of the distances, and the determining whether there exist living bodies at the distances according to the amplitude spectra and/or the phase spectra includes: determining whether there exist living bodies at the distances according to the first distributions of the amplitude spectra of the distances in a frequency and/or the second distributions of the phase spectra of the distances in a frequency.

The determining whether there exist living bodies at the distances according to first distributions of the amplitude spectra of the distances in a frequency and/or second distributions of the phase spectra of the distances in a frequency and the amplitude fluctuations of the distances may include calculating amplitude low-frequency energy ratios and/or phase low-frequency energy ratios of the distances according to the first distributions of the amplitude spectra of the distances in a frequency and/or the second distributions of the phase spectra of the distances in the frequency, and determining that there exists a living body on a distance when amplitude fluctuations of all distances in a local range centered at the distance are greater than the first threshold and at least one of the following conditions is satisfied: amplitude low-frequency energy ratios of all the distances in the local range centered at the distance are all greater than a second threshold; and phase low-frequency energy ratios of all the distances in the local range centered at the distance are all greater than a third threshold.

According to an aspect of an embodiment, when an amplitude fluctuation of the distance is of a local maximum value in the local range centered at the distance, the amplitude fluctuations of all the distances in the local range centered at the distance are greater than the first threshold, the amplitude low-frequency energy ratios of all the distances in the local range centered at the distance are all greater than the second threshold, and phase the low-frequency energy ratios of all the distances in the local range centered at the distance are all greater than the third threshold, it is determined that there exists a living body at the distance.

The determining whether there exist living bodies at the distances according to first distributions of the amplitude spectra of the distances in a frequency and/or second distributions of the phase spectra of the distances in a frequency may include calculating amplitude low-frequency energy ratios and/or phase low-frequency energy ratios of the distances according to the first distributions of the amplitude spectra of the distances in a frequency and/or the second distributions of the phase spectra of the distances in the frequency, and determining that there exists a living body at the distance when at least one of the following conditions is satisfied: amplitude low-frequency energy ratios of all the distances in the local range centered at the distance are all greater than a second threshold; and phase low-frequency energy ratios of all the distances in the local range centered at the distance are all greater than a third threshold.

According to an aspect of an embodiment, when an amplitude low-frequency energy ratio of the distance is of a local maximum value in the local range centered at the distance, the amplitude low-frequency energy ratios of all the distances in the local range centered at the distance are all greater than the second threshold, and the phase the low-frequency energy ratios of all the distances in the local range centered at the distance are all greater than the third threshold, it is determined that there exists a living body at the distance.

1. An apparatus, comprising:
a processor to couple to a memory and to,
obtain range Fast Fourier Transformation (FFT) signals within a first time range according to reflected signals of transmitted microwave signals of a microwave radar device within the first time range;
obtain amplitude distributions and/or phase distributions of distances in the first time range according to the range FFT signals within the first time range, and calculate amplitude fluctuations of the distances within the first time range;
perform Fourier transform on the amplitude distributions and/or the phase distributions to obtain amplitude spectra and/or phase spectra; and
based on magnitudes of the amplitude fluctuations, determine whether there exists a living body corresponding to the distances according to the amplitude spectra and/or the phase spectra, and the amplitude fluctuations, or determine whether there exist a living body corresponding to the distances according to the amplitude spectra and/or the phase spectra.

2. The apparatus according to claim 1, wherein to determine whether the living body exists, the processor is to:
when the amplitude fluctuations are greater than a first threshold, determine whether there exists the living body corresponding to the distances according to the amplitude spectra and/or the phase spectra, and the amplitude fluctuations; and
when the amplitude fluctuations are less than or equal to the first threshold, determine whether there exists the living body corresponding to the distances according to the amplitude spectra and/or the phase spectra.

3. The apparatus according to claim 2, wherein,
when the amplitude fluctuations are greater than the first threshold, the processor is to determine whether there exists the living body corresponding to the distances according to first distributions of the amplitude spectra of the distances in a frequency and/or second distributions of the phase spectra of the distances in a frequency, and the amplitude fluctuations of the distances;
when the amplitude fluctuations are less than or equal to the first threshold, the processor is to determine whether there exists the living body corresponding to the distances according to the first distributions of the amplitude spectra of the distances in a frequency and/or the second distributions of the phase spectra of the distances in a frequency.

4. The apparatus according to claim 3, wherein the processor to,
calculate amplitude low-frequency energy ratios and/or phase low-frequency energy ratios of the distances according to the first distributions of the amplitude spectra of the distances in a frequency and/or the second distributions of the phase spectra of the distances in the frequency; and
determine that there exists the living body corresponding to the distances, when amplitude fluctuations of a plurality of distances among the distances in a local range centered at the distance are greater than the first threshold and any one or combination of following conditions is satisfied: amplitude low-frequency energy ratios of the plurality of distances in the local range centered at the distance are greater than a second threshold, or phase low-frequency energy ratios of the plurality of distances in the local range centered at the distance are greater than a third threshold.

5. The apparatus according to claim 4, wherein,
when an amplitude fluctuation of the distance is of a local maximum value in the local range centered at the distance, the amplitude fluctuations of the plurality of distances in the local range centered at the distance are greater than the first threshold, the amplitude low-frequency energy ratios of the plurality of distances in the local range centered at the distance are all greater than the second threshold, and phase the low-frequency energy ratios of the plurality of distances in the local range centered at the distance are all greater than the third threshold, the processor is to determine that there exists the living body at the distance.

6. The apparatus according to claim 3, wherein the processor is to,
calculate the amplitude low-frequency energy ratios and/or the phase low-frequency energy ratios of the distances according to the first distribution of the amplitude spectra of the distances in a frequency and/or the second distribution of the phase spectra of the distances in a frequency; and
determine that there exists the living body corresponding to the distances when any one or combination of following conditions is satisfied: the amplitude low-frequency energy ratios of a plurality of distances among the distances in a local range centered at the distance are greater than the second threshold; and the phase low-frequency energy ratios of the plurality of distances in the local range centered at the distance are greater than the third threshold.

7. The apparatus according to claim 6, wherein,
when an amplitude low-frequency energy ratio of the distance is of a local maximum value in the local range centered at the distance, the amplitude low-frequency energy ratios of the plurality of distances in the local range centered at the distance are greater than the second threshold, and the phase the low-frequency energy ratios of the plurality of distances in the local range centered at the distance are greater than the third threshold, the processor is to determine that there exists the living body at the distance.

8. A system, comprising:
a microwave radar device comprising a signal transmitter and a signal receiver, the signal transmitter to transmit microwave signals to a space where a living body is to be located, and the signal receiver is to receive reflected signals corresponding to the transmitted microwave signals; and
the apparatus as claimed in claim 1, to perform a detection of a living body according to the reflected signals.

9. A method, comprising:
obtaining range Fast Fourier Transformation (FFT) signals within a first time range according to reflected signals of transmitted microwave signals of a microwave radar device within the first time range;
obtaining amplitude distributions and/or phase distributions of distances in the first time range according to the range FFT signals within the first time range, and calculating amplitude fluctuations of the distances within the first time range;
performing Fourier transform on the amplitude distributions and/or the phase distributions to obtain amplitude spectra and/or phase spectra; and
based on magnitudes of the amplitude fluctuations, determining whether there exists a living body corresponding to the distances according to the amplitude spectra and/or the phase spectra, and the amplitude fluctuations, or determining whether there exists a living body corresponding to the distances according to the amplitude spectra and/or the phase spectra.

10. The method according to claim 9, wherein the determining whether there exists the living body corresponding to the distances according to the amplitude spectra and/or the phase spectra, and the amplitude fluctuations, or determining whether there exists the living body corresponding to the distances according to the amplitude spectra and/or the phase spectra, comprises:
when the amplitude fluctuations are greater than a first threshold, determining whether there exists the living body corresponding to the distances according to the amplitude spectra and/or the phase spectra, and the amplitude fluctuations; and
when the amplitude fluctuations are less than or equal to the first threshold, determining whether there exists the living body corresponding to the distances according to the amplitude spectra and/or the phase spectra.
